(19) United States
(12) Patent Application Publication
(10) Pub. No.: US 2012/0157801 A1
(43) Pub. Date: Jun. 21, 2012

(54) Adaptor for On-Body Analyte Monitoring System

(75) Inventors: Udo Hoss, Castro Valley, CA (US); Craig W. Sharp, San Francisco, CA (US); Hyun Cho, Berkeley, CA (US); Todd Winkler, Cameron Park, CA (US); Paul Legg, McKinney, TX (US)

(73) Assignee: Abbott Diabetes Care Inc.

(21) Appl. No.: 13/299,119

(22) Filed: Nov. 17, 2011

Related U.S. Application Data
(60) Provisional application No. 61/415,174, filed on Nov. 18, 2010.

(51) Int. Cl. A61B 5/145

(52) U.S. Cl. 600/309

(57) ABSTRACT

An analyte monitoring system comprising: an on-body housing; an analyte sensor coupled to the housing; an electrical output interface disposed on an outer surface of the housing; and a removable adaptor coupled to the housing. In one embodiment, a portion of the analyte sensor extends from the housing for implantation into a patient's body. The electrical output interface is electrically coupled to the analyte sensor. The removable adaptor is mechanically coupled to the housing and electrically coupled to the electrical output interface. The removable adaptor serves as a data conduit between the analyte sensor and a remote device.

### CROSS-REFERENCE TO RELATED APPLICATIONS

This application claims the benefit of U.S. Provisional Application No. 61/415,174, filed on Nov. 18, 2010, which is herein incorporated by reference in its entirety.

### RELEVANT APPLICATIONS

This application is related to U.S. patent application Ser. No. 12/393,921, filed Feb. 26, 2009; U.S. patent application Ser. No. 12/807,278, filed Aug. 31, 2010; U.S. patent application Ser. No. 12/876,840, filed Sep. 7, 2010; U.S. Provisional Application No. 61/325,155, filed Apr. 16, 2010; U.S. Provisional Application No. 61/325,260, filed Apr. 16, 2010; and U.S. Provisional Application No. 61/247,519, filed Sep. 30, 2009. The disclosures of the above-mentioned applications are incorporated herein by reference in their entirety.

### BACKGROUND

Diabetes Mellitus is an incurable chronic disease in which the body does not produce or properly utilize insulin. Insulin is a hormone produced by the pancreas that regulates blood glucose. In particular, when blood glucose levels rise, e.g., after a meal, insulin lowers the blood glucose levels by facilitating blood glucose to move from the blood into the body cells. Thus, when the pancreas does not produce sufficient insulin (a condition known as Type 1 Diabetes) or does not properly utilize insulin (a condition known as Type II Diabetes), the blood glucose remains in the blood resulting in hyperglycemia or abnormally high blood sugar levels.

People suffering from diabetes often experience long-term complications. Some of these complications include blindness, kidney failure, and nerve damage. Additionally, diabetes is a factor in accelerating cardiovascular diseases such as atherosclerosis (hardening of the arteries), which often leads stroke, coronary heart disease, and other diseases, which can be life threatening.

The severity of the complications caused by both persistent high glucose levels and blood glucose level fluctuations has provided the impetus to develop diabetes management systems and treatment plans. In this regard, diabetes management plans historically included multiple daily testing of blood glucose levels typically by a finger-stick to draw and test blood. The disadvantage with finger-stick management of diabetes is that the user becomes aware of his blood glucose level only when he performs the finger-stick. Thus, blood glucose trends and blood glucose snapshots over a period of time is unknowable. More recently, diabetes management has included the implementation of glucose monitoring systems. Glucose monitoring systems have the capability to continuously monitor a user's blood glucose levels. Thus, such systems have the ability to illustrate not only present blood glucose levels but a snapshot of blood glucose levels and blood glucose fluctuations over a period of time.

### BRIEF SUMMARY

Presented herein is an analyte monitoring system including an on-body housing; an analyte sensor coupled to the housing; an electrical output interface disposed on an outer surface of the housing; and a removable adaptor coupled to the housing. In one embodiment, a portion of the analyte sensor extends from the housing for implantation into a patient's body. The electrical output interface is electrically coupled to the analyte sensor. The removable adaptor is mechanically coupled to the housing and electrically coupled to the electrical output interface. The removable adaptor serves as a data conduit between the analyte sensor and a remote device.

Certain embodiments described herein include an analyte monitoring system, including an on-body housing, an analyte sensor coupled to the housing, where a portion of the analyte sensor extends from the housing for implantation into a patient's body, an electrical output interface disposed on an outer surface of the housing, where the electrical output interface is electrically coupled to the analyte sensor; and a removable adaptor that mechanically engages with the housing and electrically couples to the electrical output interface, where the removable adaptor serves as a data conduit between the analyte sensor and a remote device.

In some embodiments, the removable adaptor includes a memory unit for logging analyte concentration data received from the implantable analyte sensor. In other embodiments, the removable adaptor includes a communications unit for transmitting data to an external receiver. For example, the communications unit transmits the data wireles sly, including via radio frequency, Bluetooth, ZigBee, infra-red, or other near-field wireless communication protocol. In some embodiments, the removable adaptor is a circular shape. In some embodiments, the removable adaptor is shaped such that its connection to the housing and electrical output interface has no orientational preference. In some embodiments, the removable adaptor includes an elongated data cord extending from the housing. For example, the elongated data cord includes a data cord output interface for direct coupling to the remote device and/or the elongated data cord includes a communications unit for wirelessly transmitting data from the analyte sensor to the remote device. In some embodiments, the data is glucose concentration data and/or ketone concentration data. In some embodiments, the removable adaptor serves as a data conduit that transmits an instantaneous data reading upon request from the remote device.

Other embodiments described herein include an analyte monitoring system, including an on-body housing, an analyte sensor coupled to the housing, where a portion of the analyte sensor extends from the housing for implantation into a patient's body, an electrical output interface disposed on an outer surface of the housing, where the electrical output interface is electrically coupled to the analyte sensor, and a removable adaptor that mechanically engages with the housing and electrically couples to the electrical output interface, where the removable adaptor serves as a data conduit between the analyte sensor and a remote device, where the removable adaptor is shaped such that its connection to the housing and electrical output interface has no orientational preference, and where the removable adaptor includes a memory unit for logging analyte concentration data received from the implantable analyte sensor, a communications unit for transmitting data to the remote device. In some embodiments, the communications unit transmits the data wirelessly, for example, via radio frequency, Bluetooth, ZigBee, infra-red, or other near-field wireless communication protocol. In some embodiments, the data is glucose concentration data and/or ketone concentration data.

Other embodiments described herein include an analyte monitoring system, including an on-body housing, an analyte sensor coupled to the housing, where a portion of the analyte sensor extends from the housing for implantation into a patient's body, an electrical output interface disposed on an outer surface of the housing, where the electrical output interface is electrically coupled to the analyte sensor, and a removable data cord that mechanically engages with the housing and electrically couples to the electrical output interface, where the data cord extends from the housing and serves as a data conduit between the analyte sensor and a remote device.

In some embodiments, the data cord includes a communications unit for transmitting data to an external receiver. In some embodiments, the communications unit transmits the data wireles sly, for example, via radio frequency, Bluetooth, ZigBee, infra-red, or other near-field wireless communication protocol. In some embodiments, the data cord includes a data cord output interface for direct coupling to the remote device. In some embodiments, the data is glucose concentration data and /or ketone concentration data. In some embodiments, the data cord serves as a data conduit that transmits an instantaneous data reading upon request from the remote device.

Other embodiments described herein include an analyte monitoring system, including an on-body housing, a self-powered analyte sensor coupled to the housing, where a portion of the analyte sensor extends from the housing for implantation into a patient's body, an electrical output interface disposed on an outer surface of the housing, where the electrical output interface is electrically coupled to the analyte sensor, and a removable adaptor that mechanically engages with the housing and electrically couples to the electrical output interface, where the removable adaptor serves as a data conduit between the analyte sensor and a remote device.

In some embodiments, the removable adaptor includes a memory unit for logging analyte concentration data received from the implantable analyte sensor. In some embodiments, the removable adaptor includes a communications unit for transmitting data to an external receiver. In some embodiments, the communications unit transmits the data wirelessly, for example, via radio frequency, Bluetooth, ZigBee, infra-red, or other near-field wireless communication protocol. In some embodiments, the removable adaptor is a circular shape. In some embodiments, the removable adaptor is shaped such that its connection to the housing and electrical output interface has no orientational preference. In some embodiments, the removable adaptor includes an elongated data cord extending from the housing. In some embodiments, the elongated data cord includes a data cord output interface for direct coupling to the remote device. In some embodiments, the elongated data cord includes a communications unit for wirelessly transmitting data from the analyte sensor to the remote device. In some embodiments, the data is glucose concentration data and/or ketone concentration data. In some embodiments, the removable adaptor serves as a data conduit that transmits an instantaneous data reading upon request from the remote device.

Other embodiments described herein include a method of preparing an analyte monitoring system, by sterilizing a self-powered analyte sensor by electron beam sterilization, coupling the analyte sensor to an on-body housing, where a portion of the analyte sensor extends from the housing for implantation into a patient's body, electrically coupling an electrical output interface disposed on an outer surface of the housing to the analyte sensor, sterilizing a removable adaptor unit with ethylene oxide, and mechanically coupling the adaptor to the housing and electrically coupling the adaptor to the electrical output interface, where the removable adaptor serves as a data conduit between the analyte sensor and a remote device.

### BRIEF DESCRIPTION OF THE FIGURES

The accompanying drawings, which are incorporated herein, form part of the specification. Together with this written description, the drawings further serve to explain the principles of, and to enable a person skilled in the relevant art(s), to make and use the present invention.

FIG. 1 illustrates a general embodiment of an analyte monitoring system.

FIG. 2A is a top view of an adaptor for use with the analyte monitoring system of FIG. 1.

FIG. 2B is a bottom view of the adaptor of FIG. 2A.

FIG. 3 is a view of an alternative adaptor for use with the analyte monitoring system of FIG. 1.

FIG. 4 is a block diagram of an analyte monitoring system according to an embodiment presented herein.

FIG. 5 is a block diagram of an embodiment of an adaptor unit of the present invention.

FIG. 6 is a block diagram of a receiver/monitor unit of the analyte monitoring system of FIG. 4.

FIG. 7 is a schematic diagram of an embodiment of an exemplary analyte sensor.

FIG. 8A shows a perspective view of an exemplary analyte sensor.

FIGS. 8B and 8C show cross sectional views of two alternative exemplary embodiments an analyte sensor.

### DETAILED DESCRIPTION OF THE INVENTION

The embodiments described herein are related to an adaptor for use with an analyte monitoring system. The adaptor provides increased functionality to an analyte monitoring system; such as, for example, ease of sterilization, logging of data in memory, selective transmission of the data, variable modes of data transmission, ease of accessing contact points, etc. Embodiments of the present invention are described in detail below. However, it is to be understood that the invention is not limited to the particular embodiments and details presented herein. Other embodiments, of course, are possible. Modifications may be made to the embodiments described herein without departing from the spirit and scope of the present invention. It is also to be understood that the detailed description provided is for the purpose of describing particular embodiments only, and is not intended to be limiting. The scope of the invention will be limited only by the appended claims.

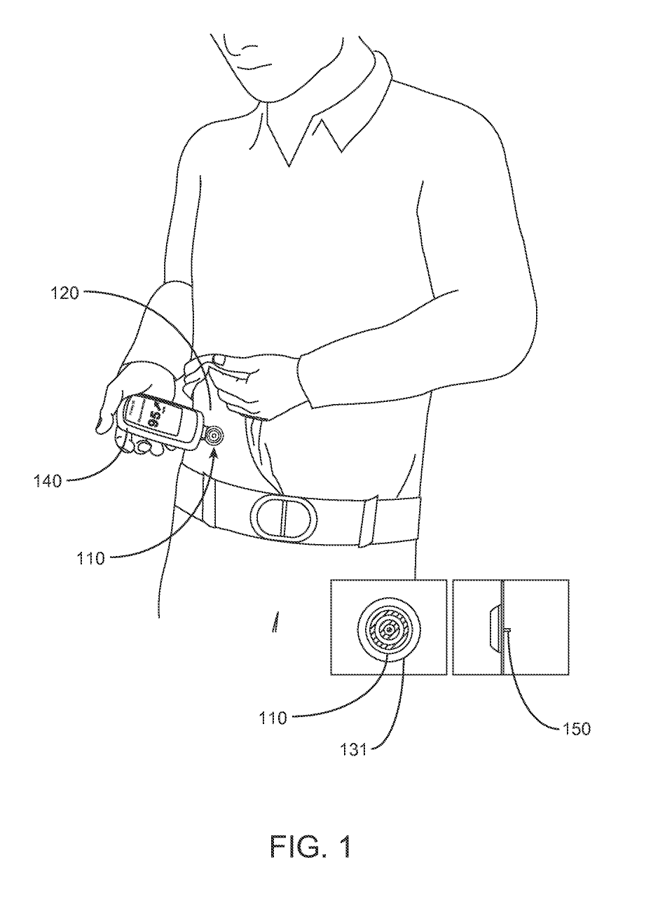

FIG. 1 illustrates a general embodiment of an analyte monitoring system. As shown, an on-body housing 110 is positioned and adhered to the skin surface 120 of the user with an adhesive 131. The right insert figure illustrates an analyte sensor 150 that may be transcutaneously positioned such that a portion of the analyte sensor is positioned and retained under the user's skin layer during the monitoring time period. The analyte sensor 150 is coupled to the on-body housing 110 such that the electrodes (working and counter electrodes, for example) of the analyte sensor 150 are electrically coupled to one or more electrical components or sensor electronics in the on-body housing 110.

While the present invention may be incorporated into battery-powered or self-powered analyte sensors, in one embodiment the analyte sensor 150 is a self-powered sensor, such as disclosed in U.S. patent application Ser. No. 12/393,921 (Publication No. 2010/0213057). When the user wishes to conduct an analyte measurement, a receiver unit (e.g., a blood glucose meter) 140 is positioned such that it electrically contacts the on-body housing 110. The contact between the on-body housing 110 with the receiver unit 140 transfers one or more signals from the electronics contained within the on-body housing 110 to the receiver unit 140. The transferred or provided signals may include signals corresponding to the real-time analyte concentration level such as, for example, real-time glucose level information; monitored analyte concentration trend information such as, for example but not limited to, the previous three hours; the rate of change of the analyte concentration determined based at least in part of the monitored analyte concentration trend information; or one or more combinations thereof.

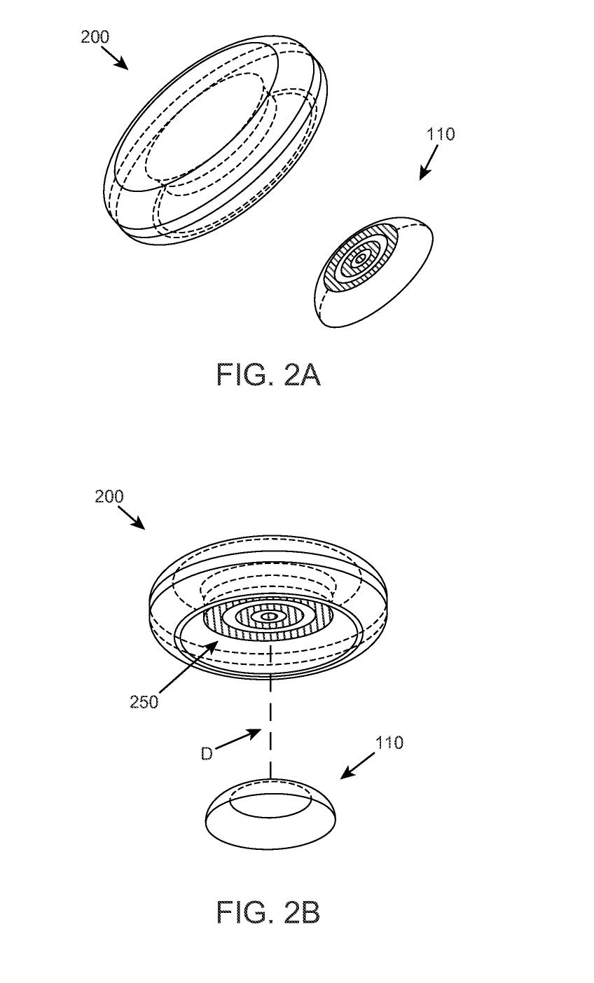

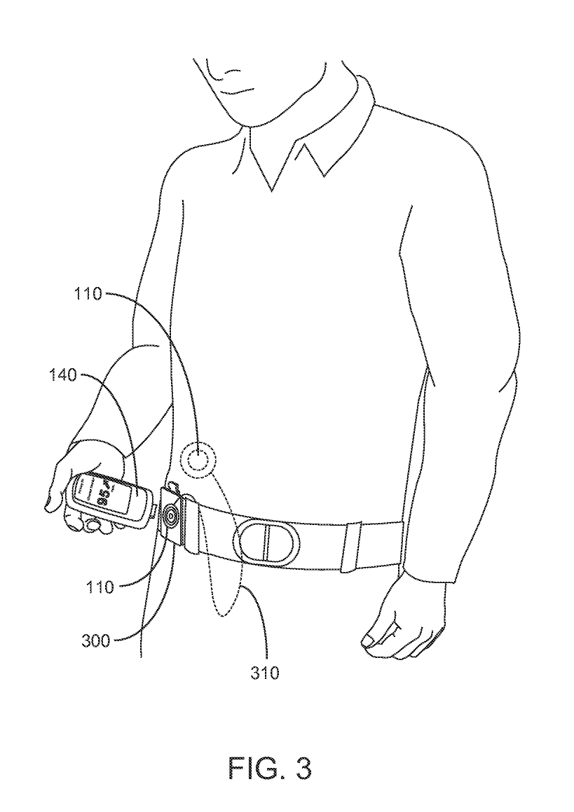

A disadvantage of the embodiment depicted in FIG. 1 is that the user must lift his clothing in order to access the on-body housing 110. Further, dirt and/or moisture may compromise the direct contact between the receiver unit 140 and the on-body housing 110. As further discussed below, FIGS. 2A, 2B, and 3 illustrate removable adaptor units to increase the functionality of the analyte monitoring system of FIG. 1.

FIG. 2A is a top view of an adaptor 200 for use with the analyte monitoring system of FIG. 1. FIG. 2B is a bottom view of the adaptor 200 of FIG. 2A. In practice, the adaptor 200 is aligned with (dotted line D) and positioned over the electrical output interface of the on-body housing 110. Concentric electrical contacts 250 are provided on the interior surface of the adaptor 200 for electrical connection with concentric electrical contacts on the on-body housing 110. As discussed below, the adaptor 200 increases the functionality of the analyte monitoring system by providing a data transfer conduit between the on-body housing 110 and a remote receiver, such as a remote analyte analysis system or meter. The adaptor 200 may also include a memory unit for programmed logging/storing of the data received from the analyte sensor 150. The adaptor 200 may also include a battery unit to power itself and/or provide power to analyte sensor 150 through on-body housing 110.

For example, in practice, the adaptor 200 is a hardware component that can be physically and electrically coupled to a sensor, e.g., a self-powered sensor, and worn on-body, along with the sensor. Through the physical and electrical coupling of the adaptor 200 and the sensor, voltages that correspond to an analyte reading will be constantly read from the sensor, and then stored in a memory unit housed within the adaptor 200. As such, the adaptor 200 may be used to convert a discrete analyte sensor system, which may have limited memory capacity and no transmitter, into a clinical diagnostic tool such as a standard continuous glucose monitoring system (CGMS).

The adaptor 200 may be used as a blind clinical diagnostic tool in which the data is stored in the adaptor and not transmitted to an external receiver. At the end of the wear cycle, the data can then be downloaded and analyzed when the adaptor is returned to a health-care professional (HCP). Alternatively, the adaptor 200 may include a transmitter, allowing data from the sensor to be transmitted to an external receiver on a pre-defined time interval via, for example, radio frequency, Bluetooth, ZigBee, infra-red, or other near-field wireless communication protocol. As such, a user or HCP may obtain continuous and/or semi-continuous glucose measurements. A battery unit within adaptor 200 may be provided to power the transmitter.

The adaptor 200 may be disposable or reusable, depending on the materials and methods used in their manufacture of the adaptor.

The modularity provided by the use of a removable adaptor also provides manufacturing advantages. For example, in practice, there are two separate sterilization techniques that are used for analyte sensors and corresponding electronics. Typically, electron beam sterilization is used for analyte sensors. Electron beam sterilization, however, is typically harmful for electronic components. As such, electronic components are sterilized with ethylene oxide. However, ethylene oxide can damage the chemistry provided on an analyte sensor. As such, integrating electronics and sensor into one unit creates manufacturing complications. However, by separating the components into a sensor unit (e.g., a self-powered analyte sensor) and adaptor unit (containing the data transmission electronics), each component can be packaged and sterilized separately using the appropriate sterilization method.

Therefore, there is provided herein a method of preparing an analyte monitoring system including: 1) sterilizing a self-powered analyte sensor by electron beam sterilization; 2) coupling the analyte sensor to an on-body housing, where a portion of the analyte sensor extends from the housing for implantation into a patient's body; 3) electrically coupling an electrical output interface disposed on an outer surface of the housing to the analyte sensor. The method further comprises: 4) sterilizing a removable adaptor unit with ethylene oxide; and 5) mechanically coupling the adaptor to the housing and electrically coupling the adaptor to the electrical output interface, where the removable adaptor serves as a data conduit between the analyte sensor and a remote device.

Using the adaptor 200 as a means of providing data storage and/or data transmission is also advantageous in that it provides more flexibility to the end-user. For example, the adaptor may be marketed as an accessory to a self-powered sensor. The sensor will provide the basic function of continuously sensing analyte levels, but customers may purchase an adaptor that would provide one ore more of the following functions: 1) blind data storage (i.e., data not visible to patient, but downloadable by a HCP) allowing the sensor to function as a blind clinical diagnostic tool; 2) transmission to an external data receiver; 3) data storage and simultaneous data transmission so that the sensor can function as a non-blind clinical diagnostic tool (continuous data is visible to patient and also stored in adaptor for later use by an HCP); or 4) semi-continuous glucose management system that provides readings to an external receiver via near-field communication (e.g., data is transmitted whenever the receiver is brought within 6-8″ of the sensor-adaptor assembly. In other words, the adaptor allows customers to customize a self-powered sensor to their individual CGM needs.

Further, adaptor 200 may be configured for “user-friendly” attachment with on-body sensor 110. For example, the adaptor and/or the on-body sensor 110 may include one or more engagement or attachment features; e.g., snap-fit engagements, latches, BNC connectors, etc. The engagement or attachment features thus serve to align and attach the adaptor 200 to the on-body sensor 110. In one embodiment, multi-directional attachment may be provided by modification of the electrical contacts and/or the housing configuration of the adaptor 200 and the on-body sensor 110. For example, discrete pin contacts may be provided on either the adaptor 200 or the on-body sensor 110 to electrically couple to the concentric circle contacts on the opposing surface of the on-body sensor 110 or adaptor 200, respectively. The housing shape of the adaptor 200 or on-body sensor 110 may also be configured to aid in the alignment and engagement between the two components. For example, in one embodiment, the on-body sensor 110 is provided with a convex shape, while the inner mating surface of the adaptor 200 is provided with a corresponding concave shape. The corresponding nature of the surfaces may provide easy engagement between the adaptor 200 and the on-body sensor 110, regardless of the direction in which the adaptor is presented to the on-body sensor 110. Any variety of corresponding housing shapes may be employed.

FIG. 3 is a view of an alternative adaptor 300 for use with the analyte monitoring system of FIG. 1. The adaptor 300 may include all the functionality of the above described adaptor 200. The adaptor 300 also includes an elongated data cord 310 that can be coupled to on-body housing 110 under the user's clothing. The elongated data cord 310 may then extend from the on-body housing 110, and provide an alternative site for data transfer to a remote device, such as receiver 140.

In practice, the data cord may be coupled at on one end (proximal) to the on-body housing 110. The other end (distal) provides a contact interface where the receiver 140 may directly connect to the cord for data transmission. Alternatively, near-field wireless protocols may be used to transfer data from the distal end of adaptor 300 to the receiver 140. Further, the distal end of the adaptor 300 may include a clip to conveniently secure the adaptor 300 the user's clothing. Alternatively, the adaptor 300 may be configured and worn like a bracelet with the data cord connecting to the insertion site of the analyte sensor. As such, the user can take analyte readings discretely by connecting the receiver 140 to the distal end of the adaptor 300, without removing the clothing at the on-body housing 110. The adaptor 300 may be disconnected when desired (e.g., at night) and reconnected any time. The adaptor 300 may be reuseable. The adaptor 300 may also include shielding to avoid noise in the signal.

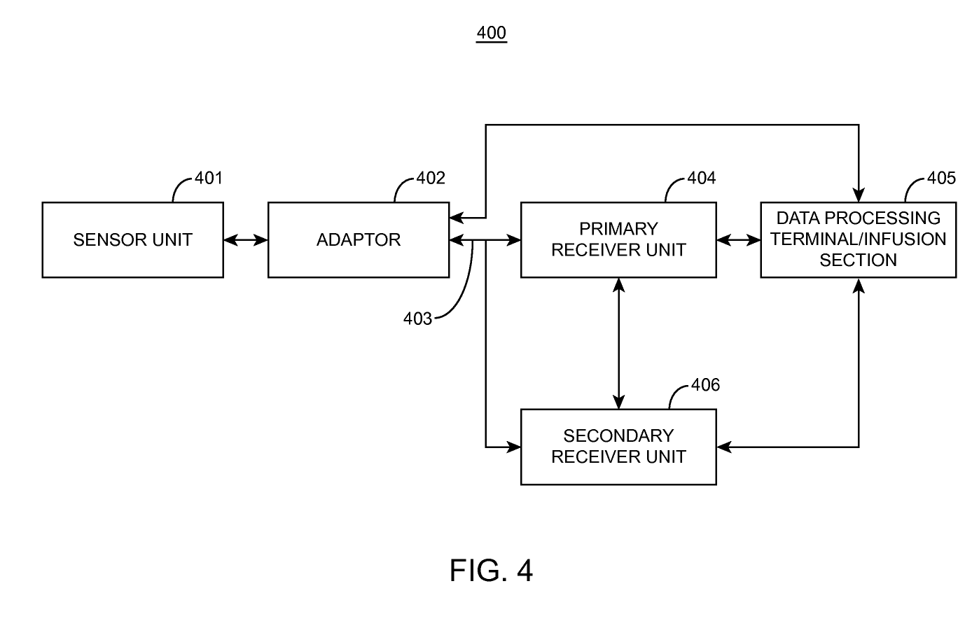

FIG. 4 shows a block diagram of an analyte monitoring system 400 according to an embodiment presented herein. Embodiments of the subject invention are further described primarily with respect to glucose monitoring devices and systems, and methods of glucose detection, for convenience only and such description is in no way intended to limit the scope of the invention. It is to be understood that the self-powered analyte monitoring system may be configured to monitor a variety of analytes at the same time or at different times.

Analytes that may be monitored include, but are not limited to, acetyl choline, amylase, bilirubin, cholesterol, chorionic gonadotropin, creatine kinase (e.g., CK-MB), creatine, creatinine, DNA, fructosamine, glucose, glutamine, growth hormones, hormones, ketone bodies, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be monitored. In those embodiments that monitor more than one analyte, the analytes may be monitored at the same or different times.

The analyte monitoring system 400 includes a sensor 401, an adaptor 402 connectable to the sensor 401, and a primary receiver unit 404 which is configured to communicate with the adaptor 402 via a communication link 403. The sensor 401 may be, for example, a self-powered analyte sensor. The adaptor 402 may be an adaptor such as described above (200 or 300), or any adaptor equivalent thereto. In certain embodiments, the primary receiver unit 404 may be further configured to transmit data to a data processing terminal 405 to evaluate or otherwise process or format data received by the primary receiver unit 404. The data processing terminal 405 may be configured to receive data directly from the adaptor 402 via a communication link which may optionally be configured for bi-directional communication. Further, the adaptor 402 may include a transmitter or a transceiver to transmit and/or receive data to and/or from the primary receiver unit 404 and/or the data processing terminal 405 and/or optionally the secondary receiver unit 406.

Also shown in FIG. 4 is an optional secondary receiver unit 406 which is operatively coupled to the communication link and configured to receive data transmitted from the adaptor 402. The secondary receiver unit 406 may be configured to communicate with the primary receiver unit 404, as well as the data processing terminal 405. The secondary receiver unit 406 may be configured for bi-directional wireless communication with each of the primary receiver unit 404 and the data processing terminal 405. As discussed in further detail below, in certain embodiments the secondary receiver unit 406 may be a de-featured receiver as compared to the primary receiver; i.e., the secondary receiver may include a limited or minimal number of functions and features as compared with the primary receiver unit 404. As such, the secondary receiver unit 406 may include a smaller (in one or more, including all, dimensions), compact housing or embodied in a device including a wrist watch, arm band, PDA, etc., for example. Alternatively, the secondary receiver unit 406 may be configured with the same or substantially similar functions and features as the primary receiver unit 404. The secondary receiver unit 406 may include a docking portion to be mated with a docking cradle unit for placement by, e.g., the bedside for night time monitoring, and/or a bi-directional communication device. A docking cradle may recharge a power supply in the secondary receiver unit 406.

Only one self-powered sensor 401, adaptor 402 and data processing terminal 405 are shown in the embodiment of the analyte monitoring system 400 illustrated in FIG. 4. However, it will be appreciated by one of ordinary skill in the art that the analyte monitoring system 400 may include more than one sensor 401 and/or more than one adaptor 402, and/or more than one data processing terminal 405. Multiple self-powered sensors may be positioned in a patient for analyte monitoring at the same or different times. In certain embodiments, analyte information obtained by a first positioned sensor may be employed as a comparison to analyte information obtained by a second sensor. This may be useful to confirm or validate analyte information obtained from one or both of the sensors. Such redundancy may be useful if analyte information is contemplated in critical therapy-related decisions. In certain embodiments, a first sensor may be used to calibrate a second sensor.

The analyte monitoring system 400 may be a continuous monitoring system, or semi-continuous, or a discrete monitoring system. In a multi-component environment, each component may be configured to be uniquely identified by one or more of the other components in the system so that communication conflict may be readily resolved between the various components within the analyte monitoring system 400. For example, unique IDs, communication channels, and the like, may be used.

In certain embodiments, the sensor 401 is physically positioned in or on the body of a user whose analyte level is being monitored. The sensor 401 may be configured to at least periodically sample the analyte level of the user and convert the sampled analyte level into a corresponding signal for transmission by the adaptor 402. The adaptor 402 is removably coupled to the self-powered sensor 401 so that both devices are positioned in or on the user's body, with at least a portion of the self-powered analyte sensor 401 positioned transcutaneously. The adaptor 402 may include a fixation element such as adhesive or the like to secure it to the sensor 401, a sensor housing, or directly to the user's body. An optional mount attachable to the user and mateable with the adaptor 402 may be used. For example, a mount may include an adhesive surface. The adaptor 402 may perform data processing functions, where such functions may include but are not limited to, filtering and encoding of data signals, each of which corresponds to a sampled analyte level of the user, for transmission to the primary receiver unit 404 via the communication link 403.

In certain embodiments, the primary receiver unit 404 may include an analog interface section including an RF receiver and an antenna that is configured to communicate with the adaptor 402 via the communication link 403, and a data processing section for processing the received data from the adaptor 402 including data decoding, error detection and correction, data clock generation, data bit recovery, etc., or any combination thereof.

In operation, the primary receiver unit 404 in certain embodiments is configured to synchronize with the adaptor 402 to uniquely identify the adaptor 402, based on, for example, an identification information of the adaptor 402, and thereafter, to periodically receive signals transmitted from the adaptor 402 associated with the monitored analyte levels detected by the sensor 401.

Referring again to FIG. 4, the data processing terminal 405 may include a personal computer, a portable computer including a laptop or a handheld device (e.g., personal digital assistants (PDAs), telephone including a cellular phone (e.g., a multimedia and Internet-enabled mobile phone including an iPhone™, or similar phone), mp3 player, pager, and the like), drug delivery device, each of which may be configured for data communication with the receiver via a wired or a wireless connection. Additionally, the data processing terminal 405 may further be connected to a data network (not shown) for storing, retrieving, updating, and/or analyzing data corresponding to the detected analyte level of the user.

The data processing terminal 405 may include an infusion device such as an insulin infusion pump or the like, which may be configured to administer insulin to patients, and which may be configured to communicate with the primary receiver unit 404 for receiving, among others, the measured analyte level. Alternatively, the primary receiver unit 404 may be configured to integrate an infusion device therein so that the primary receiver unit 404 is configured to administer insulin (or other appropriate drug) therapy to patients, for example, for administering and modifying basal profiles, as well as for determining appropriate boluses for administration based on, among others, the detected analyte levels received from the adaptor 402. An infusion device may be an external device or an internal device (wholly implantable in a user).

In certain embodiments, the data processing terminal 405, which may include an insulin pump, may be configured to receive the analyte signals from the adaptor 402, and thus, incorporate the functions of the primary receiver unit 404 including data processing for managing the patient's insulin therapy and analyte monitoring.

In certain embodiments, the communication link 403 as well as one or more of the other communication interfaces shown in FIG. 4, may use one or more of: an RF communication protocol, an infrared communication protocol, a Bluetooth enabled communication protocol, an 802.11x wireless communication protocol, or an equivalent wireless communication protocol which would allow secure, wireless communication of several units (for example, per HIPPA requirements), while avoiding potential data collision and interference.

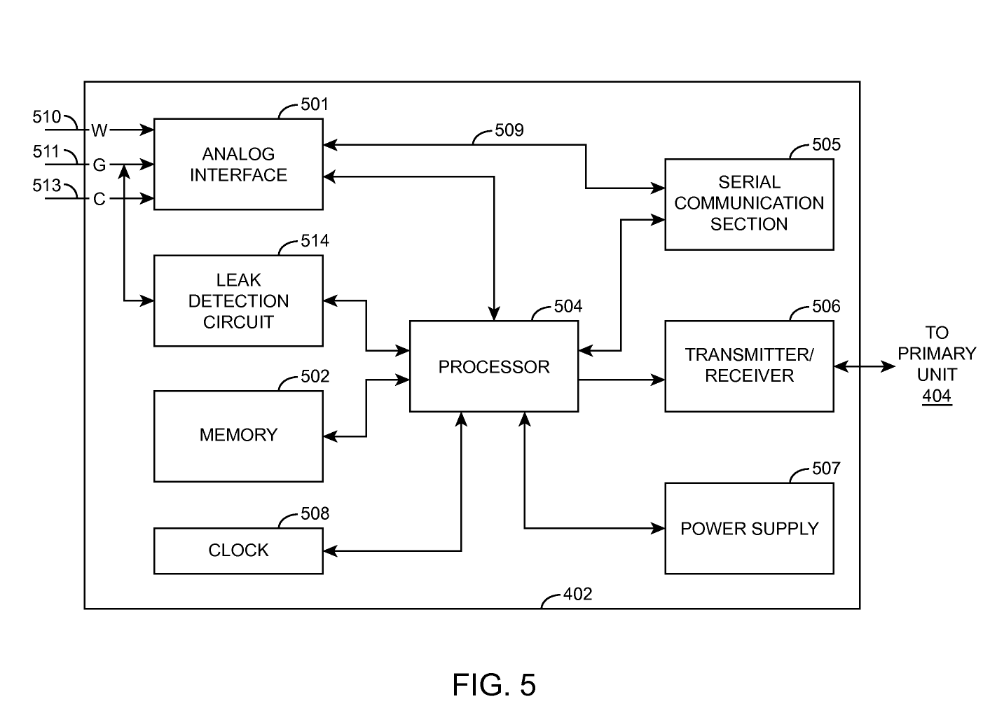

FIG. 5 shows a block diagram of an embodiment of the adaptor 402 (such as adaptor 200, adaptor 300, or equivalents thereof) of the analyte monitoring system of FIG. 4. In certain embodiments, one or more application-specific integrated circuits (ASIC) may be used to implement one or more functions or routines associated with the operations of the adaptor, using for example one or more state machines and buffers.

As can be seen in the embodiment of FIG. 5, the sensor unit 401 (FIG. 4) includes three contacts, two of which are electrodes—working electrode (W) 510, and counter electrode (C) 513, each operatively coupled to the analog interface 501 of the adaptor 402. This embodiment also shows optional guard contact (G) 511. Fewer or greater electrodes may be employed. For example, there may be more than one working electrode and/or counter electrode, etc.

In one embodiment, adaptor 402 includes a memory unit 502 for logging of the data received from sensor 401. The data may then be continuously or periodically downloaded by a HCP. By incorporating a memory unit 502 into adaptor 402, there is no need to associate a memory unit with the sensor 401. As such, the sensor 401 may be manufactured in a more efficient and cost-effective manner.

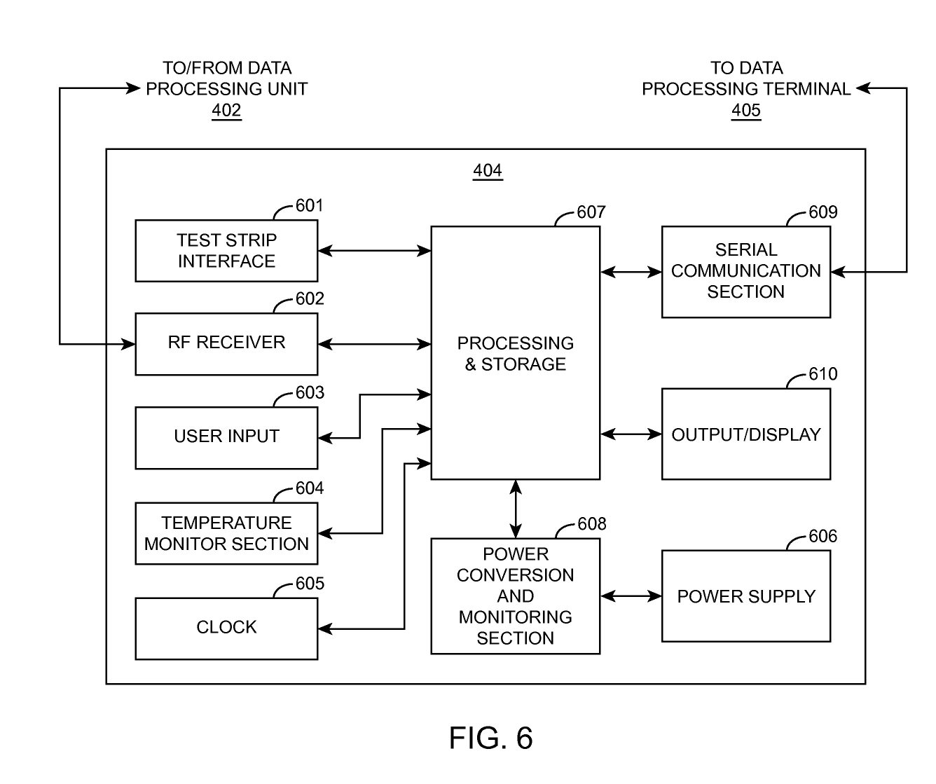

FIG. 6 shows a block diagram of a receiver/monitor unit of the analyte monitoring system of FIG. 4; such as the primary receiver unit 404. The primary receiver unit 404 may include one or more of: a blood glucose test strip interface 601 (for alternative discrete testing), an RF receiver 602, an input 603, a temperature detection section 604, and a clock 605, each of which is operatively coupled to a processing and storage section 607. The primary receiver unit 404 also includes a power supply 606 operatively coupled to a power conversion and monitoring section 608. Further, the power conversion and monitoring section 608 is also coupled to the receiver processor 607. Moreover, also shown are a receiver serial communication section 609, and an output 610, each operatively coupled to the processing and storage unit 607. The receiver may include user input and/or interface components or may be free of user input and/or interface components.

In certain embodiments, the test strip interface 601 includes a glucose level testing portion to receive a blood (or other body fluid sample) glucose test or information related thereto. For example, the interface may include a test strip port to receive a glucose test strip. The device may determine the glucose level of the test strip, and optionally display (or otherwise notice) the glucose level on the output 610 of the primary receiver unit 404. Any suitable test strip may be employed, e.g., test strips that only require a very small amount (e.g., one microliter or less, e.g., 0.5 microliter or less, e.g., 0.1 microliter or less), of applied sample to the strip in order to obtain accurate glucose information, e.g. FreeStyle® blood glucose test strips from Abbott Diabetes Care, Inc. Glucose information obtained by the in vitro glucose testing device may be used for a variety of purposes, computations, etc. For example, the information may be used to calibrate sensor 401, confirm results of the sensor 401 to increase the confidence thereof (e.g., in instances in which information obtained by sensor 401 is employed in therapy related decisions), etc.

In further embodiments, the adaptor 402 and/or the primary receiver unit 404 and/or the secondary receiver unit 405, and/or the data processing terminal/infusion section 405 may be configured to receive the blood glucose value wirelessly over a communication link from, for example, a blood glucose meter. In further embodiments, a user manipulating or using the analyte monitoring system 400 (FIG. 4) may manually input the blood glucose value using, for example, a user interface (for example, a keyboard, keypad, voice commands, and the like) incorporated in the one or more of the primary receiver unit 404, secondary receiver unit 405, or the data processing terminal/infusion section 405.

Additional embodiments are provided in U.S. Pat. Nos. 5,262,035; 5,264,104; 5,262,305; 5,320,715; 5,593,852; 6,175,752; 6,650,471; 6,746,582, and 7,811,231, each of which is incorporated herein by reference.

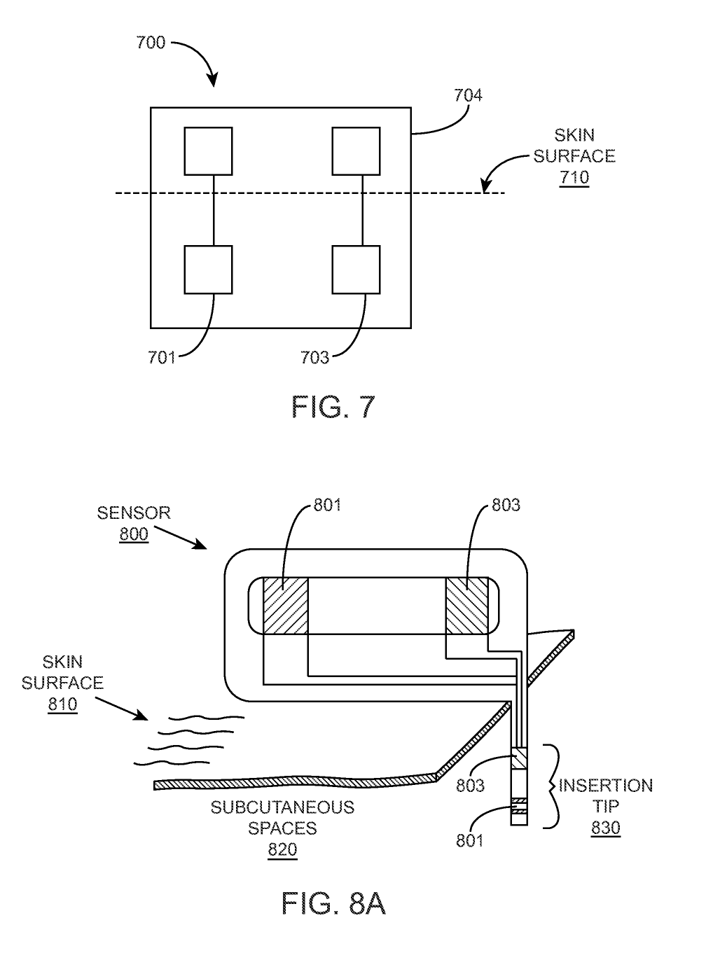

FIG. 7 shows a schematic diagram of an embodiment of an exemplary analyte sensor. This sensor embodiment includes electrodes 701 and 703 on a base 704. Electrodes (and/or other features) may be applied or otherwise processed using any suitable technology, e.g., chemical vapor deposition (CVD), physical vapor deposition, sputtering, reactive sputtering, printing, coating, ablating (e.g., laser ablation), painting, dip coating, etching, and the like. Materials include, but are not limited to, any one or more of aluminum, carbon (including graphite), cobalt, copper, gallium, gold, indium, iridium, iron, lead, magnesium, mercury (as an amalgam), nickel, niobium, osmium, palladium, platinum, rhenium, rhodium, selenium, silicon (e.g., doped polycrystalline silicon), silver, tantalum, tin, titanium, tungsten, uranium, vanadium, zinc, zirconium, mixtures thereof, and alloys, oxides, or metallic compounds of these elements.

The sensor may be wholly implantable in a user or may be configured so that only a portion is positioned within (internal) a user and another portion outside (external) a user. For example, the sensor 700 may include a portion positionable above a surface of the skin 710, and a portion positioned below the skin. In such embodiments, the external portion may include contacts (connected to respective electrodes of the second portion by traces) to connect to another device also external to the user such as a transmitter unit. While the embodiment of FIG. 7 shows two electrodes side-by-side on the same surface of base 704, other configurations are contemplated, e.g., greater electrodes, some or all electrodes on different surfaces of the base or present on another base, some or all electrodes stacked together, electrodes of differing materials and dimensions, etc.

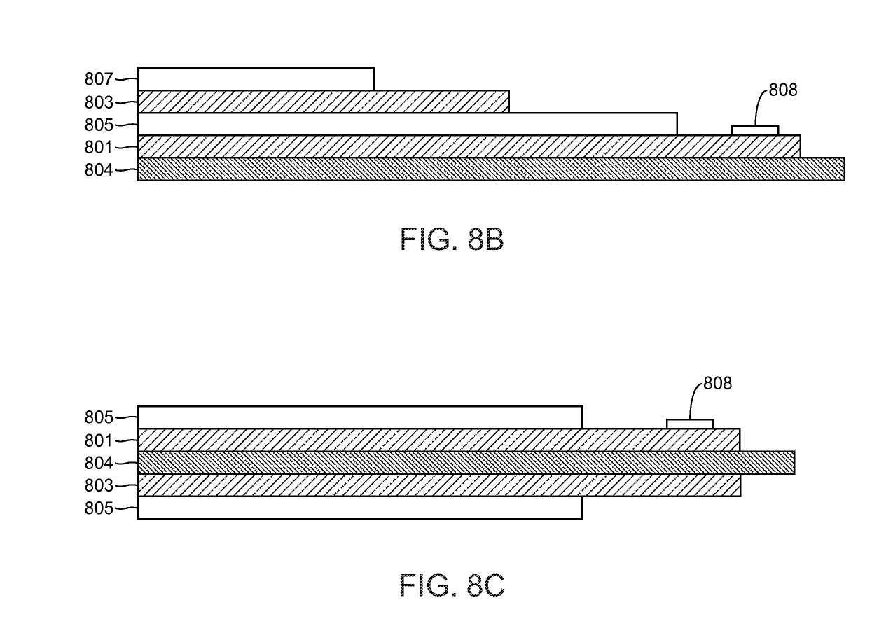

FIGS. 8A and 8B show a perspective view and a cross sectional view, respectively of another exemplary analyte sensor. More specifically, FIG. 8A shows a perspective view of an embodiment of an electrochemical analyte sensor 800 having a first portion (which in this embodiment may be characterized as a major portion) positionable above a surface of the skin 810, and a second portion (which in this embodiment may be characterized as a minor portion) that includes an insertion tip 830 positionable below the skin, e.g., penetrating through the skin and into, e.g., the subcutaneous space 820, in contact with the user's biofluid such as interstitial fluid. Contact portions of a working electrode 801 and a counter electrode 803 are positioned on the portion of the sensor 800 situated above the skin surface 810. Working electrode 801 and a counter electrode 803 are shown at the second section and particularly at the insertion tip 830. Traces may be provided from the electrode at the tip to the contact, as shown in FIG. 8A. It is to be understood that greater or fewer electrodes may be provided on a sensor. For example, a sensor may include more than one working electrodes.

FIG. 8B shows a cross sectional view of a portion of the sensor 800 of FIG. 8A. The electrodes 801 and 803 of the sensor 800 as well as the substrate and the dielectric layers are provided in a layered configuration or construction. For example, as shown in FIG. 8B, in one aspect, the sensor 800 (such as the sensor unit 401 FIG. 4), includes a substrate layer 804, and a first conducting layer 801 such as carbon, gold, etc., disposed on at least a portion of the substrate layer 804, and which may provide the working electrode. Also shown disposed on at least a portion of the first conducting layer 801 is a sensing layer 808.

A first insulation layer such as a first dielectric layer 805 is disposed or layered on at least a portion of the first conducting layer 801. A second conducting layer 803 may provide the counter electrode 803. It may be disposed on at least a portion of the first insulation layer 805. Finally, a second insulation layer may be disposed or layered on at least a portion of the second conducting layer 803. In this manner, the sensor 800 may be layered such that at least a portion of each of the conducting layers is separated by a respective insulation layer (for example, a dielectric layer). The embodiment of FIGS. 8A and 8B show the layers having different lengths. Some or all of the layers may have the same or different lengths and/or widths.

In certain embodiments, some or all of the electrodes 801, 803 may be provided on the same side of the substrate 804 in the layered construction as described above, or alternatively, may be provided in a co-planar manner such that two or more electrodes may be positioned on the same plane (e.g., side-by side (e.g., parallel) or angled relative to each other) on the substrate 804. For example, co-planar electrodes may include a suitable spacing there between and/or include dielectric material or insulation material disposed between the conducting layers/electrodes. Furthermore, as exemplified in FIG. 8C, in certain embodiments one or more of the electrodes 801, 803 may be disposed on opposing sides of the substrate 804. In such embodiments, contact pads may be one the same or different sides of the substrate. For example, an electrode may be on a first side and its respective contact may be on a second side, e.g., a trace connecting the electrode and the contact may traverse through the substrate.

As noted above, analyte sensors may include an analyte-responsive enzyme to provide a sensing component or sensing layer. Some analytes, such as oxygen, can be directly electrooxidized or electroreduced on a sensor, and more specifically at least on a working electrode of a sensor. Other analytes, such as glucose and lactate, require the presence of at least one electron transfer agent and/or at least one catalyst to facilitate the electrooxidation or electroreduction of the analyte. Catalysts may also be used for those analytes, such as oxygen, that can be directly electrooxidized or electroreduced on the working electrode. For these analytes, each working electrode includes a sensing layer (see for example sensing layer 808 of FIG. 8B) proximate to or on a surface of a working electrode. In many embodiments, a sensing layer is formed near or on only a small portion of at least a working electrode.

The sensing layer includes one or more components constructed to facilitate the electrochemical oxidation or reduction of the analyte. The sensing layer may include, for example, a catalyst to catalyze a reaction of the analyte and produce a response at the working electrode, an electron transfer agent to transfer electrons between the analyte and the working electrode (or other component), or both. The sensing layer and the working electrode also function as the anode of the power generating component of the self-powered analyte sensor, thereby providing the dual-function of power generation and analyte level detection.

A variety of different sensing layer configurations may be used. In certain embodiments, the sensing layer is deposited on the conductive material of a working electrode. The sensing layer may extend beyond the conductive material of the working electrode. In some cases, the sensing layer may also extend over other electrodes.

A sensing layer that is in direct contact with the working electrode may contain an electron transfer agent to transfer electrons directly or indirectly between the analyte and the working electrode, and/or a catalyst to facilitate a reaction of the analyte. For example, a glucose, lactate, or oxygen electrode may be formed having a sensing layer which contains a catalyst, including glucose oxidase, glucose dehydrogenase, lactate oxidase, or laccase, respectively, and an electron transfer agent that facilitates the electrooxidation of the glucose, lactate, or oxygen, respectively.

In other embodiments the sensing layer is not deposited directly on the working electrode. Instead, the sensing layer 808 may be spaced apart from the working electrode, and separated from the working electrode, e.g., by a separation layer. A separation layer may include one or more membranes or films or a physical distance. In addition to separating the working electrode from the sensing layer the separation layer may also act as a mass transport limiting layer and/or an interferent eliminating layer and/or a biocompatible layer.

In certain embodiments, which include more than one working electrode, one or more of the working electrodes may not have a corresponding sensing layer, or may have a sensing layer which does not contain one or more components (e.g., an electron transfer agent and/or catalyst) needed to electrolyze the analyte. Thus, the signal at this working electrode may correspond to background signal which may be removed from the analyte signal obtained from one or more other working electrodes that are associated with fully-functional sensing layers by, for example, subtracting the signal.

In certain embodiments, the sensing layer includes one or more electron transfer agents. Electron transfer agents that may be employed are electro-reducible and electro-oxidizable ions or molecules having redox potentials that are a few hundred millivolts above or below the redox potential of the standard calomel electrode (SCE). The electron transfer agent may be organic, organometallic, or inorganic. Examples of organic redox species are quinones and species that in their oxidized state have quinoid structures, such as Nile blue and indophenol. Examples of organometallic redox species are metallocenes including ferrocene. Examples of inorganic redox species are hexacyanoferrate (III), ruthenium hexamine etc. Additional examples include those described in U.S. Pat. No. 6,736,957 and U.S. Patent Publication Nos. 2004/0079653 and 2006/0201805, the disclosures of which are incorporated herein by reference in their entirety.

In certain embodiments, electron transfer agents have structures or charges which prevent or substantially reduce the diffusional loss of the electron transfer agent during the period of time that the sample is being analyzed. For example, electron transfer agents include but are not limited to a redox species, e.g., bound to a polymer which can in turn be disposed on or near the working electrode. The bond between the redox species and the polymer may be covalent, coordinative, or ionic. Although any organic, organometallic or inorganic redox species may be bound to a polymer and used as an electron transfer agent, in certain embodiments the redox species is a transition metal compound or complex, e.g., osmium, ruthenium, iron, and cobalt compounds or complexes. It will be recognized that many redox species described for use with a polymeric component may also be used, without a polymeric component.

One type of polymeric electron transfer agent contains a redox species covalently bound in a polymeric composition. An example of this type of mediator is poly(vinylferrocene). Another type of electron transfer agent contains an ionically-bound redox species. This type of mediator may include a charged polymer coupled to an oppositely charged redox species. Examples of this type of mediator include a negatively charged polymer coupled to a positively charged redox species such as an osmium or ruthenium polypyridyl cation. Another example of an ionically-bound mediator is a positively charged polymer including quaternized poly(4-vinyl pyridine) or poly(1-vinyl imidazole) coupled to a negatively charged redox species such as ferricyanide or ferrocyanide. In other embodiments, electron transfer agents include a redox species coordinatively bound to a polymer. For example, the mediator may be formed by coordination of an osmium or cobalt 2,2′-bipyridyl complex to poly(1-vinyl imidazole) or poly(4-vinyl pyridine).

Suitable electron transfer agents are osmium transition metal complexes with one or more ligands, each ligand having a nitrogen-containing heterocycle such as 2,2′-bipyridine, 1,10-phenanthroline, 1-methyl, 2-pyridyl biimidazole, or derivatives thereof. The electron transfer agents may also have one or more ligands covalently bound in a polymer, each ligand having at least one nitrogen-containing heterocycle, such as pyridine, imidazole, or derivatives thereof. One example of an electron transfer agent includes (a) a polymer or copolymer having pyridine or imidazole functional groups and (b) osmium cations complexed with two ligands, each ligand containing 2,2′-bipyridine, 1,10-phenanthroline, or derivatives thereof, the two ligands not necessarily being the same. Some derivatives of 2,2′-bipyridine for complexation with the osmium cation include but are not limited to 4,4′-dimethyl-2,2′-bipyridine and mono-, di-, and polyalkoxy-2,2′-bipyridines, including 4,4′-dimethoxy-2,2′-bipyridine. Derivatives of 1,10-phenanthroline for complexation with the osmium cation include but are not limited to 4,7-dimethyl-1,10-phenanthroline and mono, di-, and polyalkoxy-1,10-phenanthrolines, such as 4,7-dimethoxy-1,10-phenanthroline. Polymers for complexation with the osmium cation include but are not limited to polymers and copolymers of poly(1-vinyl imidazole) (referred to as “PVI”) and poly(4-vinyl pyridine) (referred to as “PVP”). Suitable copolymer substituents of poly(1-vinyl imidazole) include acrylonitrile, acrylamide, and substituted or quaternized N-vinyl imidazole, e.g., electron transfer agents with osmium complexed to a polymer or copolymer of poly(l-vinyl imidazole).

Embodiments may employ electron transfer agents having a redox potential ranging from about −200 mV to about +200 mV versus the standard calomel electrode (SCE). The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase, flavine adenine dinucleotide (FAD) dependent glucose dehydrogenase, or nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

The sensing layer may also include a catalyst which is capable of catalyzing a reaction of the analyte. The catalyst may also, in some embodiments, act as an electron transfer agent. One example of a suitable catalyst is an enzyme which catalyzes a reaction of the analyte. For example, a catalyst, including a glucose oxidase, glucose dehydrogenase (e.g., pyrroloquinoline quinone (PQQ), dependent glucose dehydrogenase or oligosaccharide dehydrogenase, flavine adenine dinucleotide (FAD) dependent glucose dehydrogenase, nicotinamide adenine dinucleotide (NAD) dependent glucose dehydrogenase), may be used when the analyte of interest is glucose. A lactate oxidase or lactate dehydrogenase may be used when the analyte of interest is lactate. Laccase may be used when the analyte of interest is oxygen or when oxygen is generated or consumed in response to a reaction of the analyte.

In certain embodiments, a catalyst may be attached to a polymer, cross linking the catalyst with another electron transfer agent, which, as described above, may be polymeric. A second catalyst may also be used in certain embodiments. This second catalyst may be used to catalyze a reaction of a product compound resulting from the catalyzed reaction of the analyte. The second catalyst may operate with an electron transfer agent to electrolyze the product compound to generate a signal at the working electrode. Alternatively, a second catalyst may be provided in an interferent-eliminating layer to catalyze reactions that remove interferents.

In certain embodiments, the sensing layer functions at a gentle oxidizing potential, e.g., a potential of about +40 mV vs. Ag/AgCl. This sensing layer uses, for example, an osmium (Os)-based mediator constructed for low potential operation and includes a plasticizer. Accordingly, in certain embodiments the sensing element is a redox active component that includes (1) Osmium-based mediator molecules that include (bidente) ligands, and (2) glucose oxidase enzyme molecules. These two constituents are combined together with a cross-linker.

A mass transport limiting layer (not shown), e.g., an analyte flux modulating layer, may be included with the sensor to act as a diffusion-limiting barrier to reduce the rate of mass transport of the analyte, for example, glucose or lactate, into the region around the working electrodes. The mass transport limiting layers are useful in limiting the flux of an analyte to a working electrode in an electrochemical sensor so that the sensor is linearly responsive over a large range of analyte concentrations and is easily calibrated. Mass transport limiting layers may include polymers and may be biocompatible. A mass transport limiting layer may provide many functions, e.g., biocompatibility and/or interferent-eliminating, etc.

In certain embodiments, a mass transport limiting layer is a membrane composed of crosslinked polymers containing heterocyclic nitrogen groups, such as polymers of polyvinylpyridine and polyvinylimidazole. In some embodiments, a plasticizer is combined with the mass transport limiting layer or membrane. Embodiments also include membranes that are made of a polyurethane, or polyether urethane, or chemically related material, or membranes that are made of silicone, and the like.

A membrane may be formed by crosslinking in situ a polymer, modified with a zwitterionic moiety, a non-pyridine copolymer component, and optionally another moiety that is either hydrophilic or hydrophobic, and/or has other desirable properties, in an alcohol-buffer solution. In certain embodiments, the membrane formulation further includes a plasticizer. The modified polymer may be made from a precursor polymer containing heterocyclic nitrogen groups. For example, a precursor polymer may be polyvinylpyridine or polyvinylimidazole. Optionally, hydrophilic or hydrophobic modifiers may be used to “fine-tune” the permeability of the resulting membrane to an analyte of interest. Optional hydrophilic modifiers, such as poly(ethylene glycol), hydroxyl or polyhydroxyl modifiers, may be used to enhance the biocompatibility of the polymer or the resulting membrane.

A membrane may be formed in situ by applying an alcohol-buffer solution of a crosslinker and a modified polymer over an enzyme-containing sensing layer and allowing the solution to cure for about one to two days or other appropriate time period. The crosslinker-polymer solution may be applied to the sensing layer by placing a droplet or droplets of the solution on the sensor, by dipping the sensor into the solution, or the like. Generally, the thickness of the membrane is controlled by the concentration of the solution, by the number of droplets of the solution applied, by the number of times the sensor is dipped in the solution, or by any combination of these factors. A membrane applied in this manner may have any combination of the following functions: (1) mass transport limitation, i.e., reduction of the flux of analyte that can reach the sensing layer, (2) biocompatibility enhancement, or (3) interferent reduction.

The substrate may be formed using a variety of non-conducting materials, including, for example, polymeric or plastic materials and ceramic materials. Suitable materials for a particular sensor may be determined, at least in part, based on the desired use of the sensor and properties of the materials.

In some embodiments, the substrate is flexible. For example, if the sensor is configured for implantation into a patient, then the sensor may be made flexible (although rigid sensors may also be used for implantable sensors) to reduce pain to the patient and damage to the tissue caused by the implantation of and/or the wearing of the sensor. A flexible substrate often increases the patient's comfort and allows a wider range of activities. Suitable materials for a flexible substrate include, for example, non-conducting plastic or polymeric materials and other non-conducting, flexible, deformable materials. Examples of useful plastic or polymeric materials include thermoplastics such as polycarbonates, polyesters (e.g., Mylar™ and polyethylene terephthalate (PET)), polyvinyl chloride (PVC), polyurethanes, polyethers, polyamides, polyimides, or copolymers of these thermoplastics, such as PETG (glycol-modified polyethylene terephthalate).

In other embodiments, the sensors are made using a relatively rigid substrate to, for example, provide structural support against bending or breaking. Examples of rigid materials that may be used as the substrate include poorly conducting ceramics, such as aluminum oxide and silicon dioxide. One advantage of an implantable sensor having a rigid substrate is that the sensor may have a sharp point and/or a sharp edge to aid in implantation of a sensor without an additional insertion device.

It will be appreciated that for many sensors and sensor applications, both rigid and flexible sensors will operate adequately. The flexibility of the sensor may also be controlled and varied along a continuum by changing, for example, the composition and/or thickness of the substrate.

In addition to considerations regarding flexibility, it is often desirable that implantable sensors should have a substrate which is physiologically harmless, for example, a substrate approved by a regulatory agency or private institution for in vivo use.

The sensor may include optional features to facilitate insertion of an implantable sensor. For example, the sensor may be pointed at the tip to ease insertion. In addition, the sensor may include a barb which assists in anchoring the sensor within the tissue of the patient during operation of the sensor. However, the barb is typically small enough so that little damage is caused to the subcutaneous tissue when the sensor is removed for replacement.

An implantable sensor may also, optionally, have an anticlotting agent disposed on a portion of the substrate which is implanted into a patient. This anticlotting agent may reduce or eliminate the clotting of blood or other body fluid around the sensor, particularly after insertion of the sensor. Blood clots may foul the sensor or irreproducibly reduce the amount of analyte which diffuses into the sensor. Examples of useful anticlotting agents include heparin and tissue plasminogen activator (TPA), as well as other known anticlotting agents.

The anticlotting agent may be applied to at least a portion of that part of the sensor that is to be implanted. The anticlotting agent may be applied, for example, by bath, spraying, brushing, or dipping. The anticlotting agent is allowed to dry on the sensor. The anticlotting agent may be immobilized on the surface of the sensor or it may be allowed to diffuse away from the sensor surface. Typically, the quantities of anticlotting agent disposed on the sensor are far below the amounts typically used for treatment of medical conditions involving blood clots and, therefore, have only a limited, localized effect.

### Insertion Device

An insertion device can be used to subcutaneously insert the self-powered analyte sensor into the patient. The insertion device is typically formed using structurally rigid materials, such as metal or rigid plastic. Exemplary materials include stainless steel and ABS (acrylonitrile-butadiene-styrene) plastic. In some embodiments, the insertion device is pointed and/or sharp at the tip to facilitate penetration of the skin of the patient. A sharp, thin insertion device may reduce pain felt by the patient upon insertion of the self-powered analyte sensor. In other embodiments, the tip of the insertion device has other shapes, including a blunt or flat shape. These embodiments may be particularly useful when the insertion device does not penetrate the skin but rather serves as a structural support for the sensor as the sensor is pushed into the skin.

### Sensor Control Unit

The sensor control unit can be integrated in the sensor, part or all of which is subcutaneously implanted or it can be configured to be placed on the skin of a patient. The sensor control unit is optionally formed in a shape that is comfortable to the patient and which may permit concealment, for example, under a patient's clothing. The thigh, leg, upper arm, shoulder, or abdomen are convenient parts of the patient's body for placement of the sensor control unit to maintain concealment. However, the sensor control unit may be positioned on other portions of the patient's body. One embodiment of the sensor control unit has a thin, oval shape to enhance concealment. However, other shapes and sizes may be used.

The particular profile, as well as the height, width, length, weight, and volume of the sensor control unit may vary and depends, at least in part, on the components and associated functions included in the sensor control unit. In general, the sensor control unit includes a housing typically formed as a single integral unit that rests on the skin of the patient. The housing typically contains most or all of the electronic components of the sensor control unit.

The housing of the sensor control unit may be formed using a variety of materials, including, for example, plastic and polymeric materials, particularly rigid thermoplastics and engineering thermoplastics. Suitable materials include, for example, polyvinyl chloride, polyethylene, polypropylene, polystyrene, ABS polymers, and copolymers thereof. The housing of the sensor control unit may be formed using a variety of techniques including, for example, injection molding, compression molding, casting, and other molding methods. Hollow or recessed regions may be formed in the housing of the sensor control unit. The electronic components of the sensor control unit and/or other items, including a battery or a speaker for an audible alarm, may be placed in the hollow or recessed areas.

The sensor control unit is typically attached to the skin of the patient, for example, by adhering the sensor control unit directly to the skin of the patient with an adhesive provided on at least a portion of the housing of the sensor control unit which contacts the skin or by suturing the sensor control unit to the skin through suture openings in the sensor control unit.

When positioned on the skin of a patient, the sensor and the electronic components within the sensor control unit are coupled via conductive contacts. The one or more working electrodes, counter electrode, and optional temperature probe are attached to individual conductive contacts. For example, the conductive contacts are provided on the interior of the sensor control unit. Other embodiments of the sensor control unit have the conductive contacts disposed on the exterior of the housing. The placement of the conductive contacts is such that they are in contact with the contact pads on the sensor when the sensor is properly positioned within the sensor control unit.

### Sensor Control Unit Electronics

The sensor control unit also typically includes at least a portion of the electronic components that measure the sensor current and the analyte monitoring device system. The electronic components of the sensor control unit typically include a power supply for operating the sensor control unit, a sensor circuit for obtaining signals from the sensor, a measurement circuit that converts sensor signals to a desired format, and a processing circuit that, at minimum, obtains signals from the sensor circuit and/or measurement circuit and provides the signals to an optional transmitter. In some embodiments, the processing circuit may also partially or completely evaluate the signals from the sensor and convey the resulting data to the optional transmitter and/or activate an optional alarm system if the analyte level exceeds a threshold. The processing circuit often includes digital logic circuitry.

The sensor control unit may optionally contain a transmitter for transmitting the sensor signals or processed data from the processing circuit to a receiver/display unit; a data storage unit for temporarily or permanently storing data from the processing circuit; a temperature probe circuit for receiving signals from and operating a temperature probe; a reference voltage generator for providing a reference voltage for comparison with sensor-generated signals; and/or a watchdog circuit that monitors the operation of the electronic components in the sensor control unit.

Moreover, the sensor control unit may also include digital and/or analog components utilizing semiconductor devices, including transistors. To operate these semiconductor devices, the sensor control unit may include other components including, for example, a bias control generator to correctly bias analog and digital semiconductor devices, an oscillator to provide a clock signal, and a digital logic and timing component to provide timing signals and logic operations for the digital components of the circuit.

As an example of the operation of these components, the sensor circuit and the optional temperature probe circuit provide raw signals from the sensor to the measurement circuit. The measurement circuit converts the raw signals to a desired format, using for example, a current-to-voltage converter, current-to-frequency converter, and/or a binary counter or other indicator that produces a signal proportional to the absolute value of the raw signal. This may be used, for example, to convert the raw signal to a format that can be used by digital logic circuits. The processing circuit may then, optionally, evaluate the data and provide commands to operate the electronics.

### Calibration

Sensors may be configured to require no system calibration or no user calibration. For example, a sensor may be factory calibrated and need not require further calibrating by the user during use of the sensor. In certain embodiments, calibration may be required, but may be done without user intervention, i.e., may be automatic. In those embodiments in which calibration by the user is required, the calibration may be according to a predetermined schedule or may be dynamic, i.e., the time for which may be determined by the system on a real-time basis according to various factors, including, but not limited to, glucose concentration and/or temperature and/or rate of change of glucose, etc.

In addition to a transmitter, an optional receiver may be included in the sensor control unit. In some cases, the transmitter is a transceiver, operating as both a transmitter and a receiver. The receiver may be used to receive calibration data for the sensor. The calibration data may be used by the processing circuit to correct signals from the sensor. This calibration data may be transmitted by the receiver/display unit or from some other source such as a control unit in a doctor's office. In addition, the optional receiver may be used to receive a signal from the receiver/display units to direct the transmitter, for example, to change frequencies or frequency bands, to activate or deactivate the optional alarm system and/or to direct the transmitter to transmit at a higher rate.

Calibration data may be obtained in a variety of ways. For instance, the calibration data may simply be factory-determined calibration measurements which can be input into the sensor control unit using the receiver or may alternatively be stored in a calibration data storage unit within the sensor control unit itself (in which case a receiver may not be needed). The calibration data storage unit may be, for example, a readable or readable/writeable memory circuit.

Calibration may be accomplished using an in vitro test strip (or other reference), e.g., a small sample test strip such as a test strip that requires less than about 1 microliter of sample (for example FreeStyle® blood glucose monitoring test strips from Abbott Diabetes Care). For example, test strips that require less than about 1 nanoliter of sample may be used. In certain embodiments, a sensor may be calibrated using only one sample of body fluid per calibration event. For example, a user need only lance a body part one time to obtain sample for a calibration event (e.g., for a test strip), or may lance more than one time within a short period of time if an insufficient volume of sample is firstly obtained. Embodiments include obtaining and using multiple samples of body fluid for a given calibration event, where glucose values of each sample are substantially similar. Data obtained from a given calibration event may be used independently to calibrate or combined with data obtained from previous calibration events, e.g., averaged including weighted averaged, etc., to calibrate. In certain embodiments, a system need only be calibrated once by a user, where recalibration of the system is not required.

Alternative or additional calibration data may be provided based on tests performed by a doctor or some other professional or by the patient. For example, it is common for diabetic individuals to determine their own blood glucose concentration using commercially available testing kits. The results of this test is input into the sensor control unit either directly, if an appropriate input device (e.g., a keypad, an optical signal receiver, or a port for connection to a keypad or computer) is incorporated in the sensor control unit, or indirectly by inputting the calibration data into the receiver/display unit and transmitting the calibration data to the sensor control unit.

Other methods of independently determining analyte levels may also be used to obtain calibration data. This type of calibration data may supplant or supplement factory-determined calibration values.

In some embodiments of the invention, calibration data may be required at periodic intervals, for example, every eight hours, once a day, or once a week, to confirm that accurate analyte levels are being reported. Calibration may also be required each time a new sensor is implanted or if the sensor exceeds a threshold minimum or maximum value or if the rate of change in the sensor signal exceeds a threshold value. In some cases, it may be necessary to wait a period of time after the implantation of the sensor before calibrating to allow the sensor to achieve equilibrium. In some embodiments, the sensor is calibrated only after it has been inserted. In other embodiments, no calibration of the sensor is needed.

### Analyte Monitoring Device

In some embodiments of the invention, the analyte monitoring device includes a sensor control unit and a self-powered analyte sensor. In some embodiments, the processing circuit of the sensor control unit is able to determine a level of the analyte and activate an alarm system if the analyte level exceeds a threshold. The sensor control unit, in these embodiments, has an alarm system and may also include a display, such as an LCD or LED display.

A threshold value is exceeded if the datapoint has a value that is beyond the threshold value in a direction indicating a particular condition. For example, a datapoint which correlates to a glucose level of 200 mg/dL exceeds a threshold value for hyperglycemia of 180 mg/dL, because the datapoint indicates that the patient has entered a hyperglycemic state. As another example, a datapoint which correlates to a glucose level of 65 mg/dL exceeds a threshold value for hypoglycemia of 70 mg/dL because the datapoint indicates that the patient is hypoglycemic as defined by the threshold value. However, a datapoint which correlates to a glucose level of 75 mg/dL would not exceed the same threshold value for hypoglycemia because the datapoint does not indicate that particular condition as defined by the chosen threshold value.

An alarm may also be activated if the sensor readings indicate a value that is beyond a measurement range of the sensor. For glucose, the physiologically relevant measurement range is typically about 30 to 500 mg/dL, including about 40-300 mg/dL and about 50-250 mg/dL, of glucose in the interstitial fluid.

The alarm system may also, or alternatively, be activated when the rate of change or acceleration of the rate of change in analyte level increase or decrease reaches or exceeds a threshold rate or acceleration. For example, in the case of a subcutaneous glucose monitor, the alarm system might be activated if the rate of change in glucose concentration exceeds a threshold value which might indicate that a hyperglycemic or hypoglycemic condition is likely to occur.

A system may also include system alarms that notify a user of system information such as battery condition, calibration, sensor dislodgment, sensor malfunction, etc. Alarms may be, for example, auditory and/or visual. Other sensory-stimulating alarm systems may be used including alarm systems which heat, cool, vibrate, or produce a mild electrical shock when activated.

### Drug Delivery System

The subject invention also includes sensors used in sensor-based drug delivery systems. The system may provide a drug to counteract the high or low level of the analyte in response to the signals from one or more sensors. Alternatively, the system may monitor the drug concentration to ensure that the drug remains within a desired therapeutic range. The drug delivery system may include one or more (e.g., two or more) sensors, a processing unit such as a transmitter, a receiver/display unit, and a drug administration system. In some cases, some or all components may be integrated in a single unit. A sensor-based drug delivery system may use data from the one or more sensors to provide necessary input for a control algorithm/mechanism to adjust the administration of drugs, e.g., automatically or semi-automatically. As an example, a glucose sensor may be used to control and adjust the administration of insulin from an external or implanted insulin pump.

Each of the various references, presentations, publications, provisional and/or non-provisional U.S. Patent Applications, U.S. patents, non-U.S. Patent Applications, and/or non-U.S. patents that have been identified herein, is incorporated herein in its entirety by this reference.

Other aspects, advantages, and modifications within the scope of the invention will be apparent to those skilled in the art to which the invention pertains. Various modifications, processes, as well as numerous structures to which the embodiments of the invention may be applicable will be readily apparent to those of skill in the art to which the invention is directed upon review of the specification. Various aspects and features of the invention may have been explained or described in relation to understandings, beliefs, theories, underlying assumptions, and/or working or prophetic examples, although it will be understood that the invention is not bound to any particular understanding, belief, theory, underlying assumption, and/or working or prophetic example. Although various aspects and features of the invention may have been described largely with respect to applications, or more specifically, medical applications, involving diabetic humans, it will be understood that such aspects and features also relate to any of a variety of applications involving non-diabetic humans and any and all other animals. Further, although various aspects and features of the invention may have been described largely with respect to applications involving partially implanted sensors, such as transcutaneous or subcutaneous sensors, it will be understood that such aspects and features also relate to any of a variety of sensors that are suitable for use in connection with the body of an animal or a human, such as those suitable for use as fully implanted in the body of an animal or a human. Finally, although the various aspects and features of the invention have been described with respect to various embodiments and specific examples herein, all of which may be made or carried out conventionally, it will be understood that the invention is entitled to protection within the full scope of the appended claims.

### Calculation of Medication Dosage

In one embodiment, the analyte measurement system may be configured to measure the blood glucose concentration of a patient and include instructions for a long-acting insulin dosage calculation function. Periodic injection or administration of long-acting insulin may be used to maintain a baseline blood glucose concentration in a patient with Type-1 or Type-2 diabetes. In one aspect, the long-acting medication dosage calculation function may include an algorithm or routine based on the current blood glucose concentration of a diabetic patient, to compare the current measured blood glucose concentration value to a predetermined threshold or an individually tailored threshold as determined by a doctor or other treating professional to determine the appropriate dosage level for maintaining the baseline glucose level. In one embodiment, the long-acting insulin dosage calculation function may be based upon LANTUS® insulin, available from Sanofi-Aventis, also known as insulin glargine. LANTUS® is a long-acting insulin that has up to a 24 hour duration of action. Further information on LANTUS® insulin is available at the website located by placing “www” immediately in front of “.lantus.com”. Other types of long-acting insulin include Levemir® insulin available from NovoNordisk (further information is available at the website located by placing “www” immediately in front of “.levemir-us.com”. Examples of such embodiments are described in US Published Patent Application No. US2010/01981142, the disclosure of which is incorporated herein by reference in its entirety.

Integration with Medication Delivery Devices and/or Systems

In some embodiments, the analyte measurement systems disclosed herein may be included in and/or integrated with, a medication delivery device and/or system, e.g., an insulin pump module, such as an insulin pump or controller module thereof. In some embodiments the analyte measurement system is physically integrated into a medication delivery device. In other embodiments, an analyte measurement system as described herein may be configured to communicate with a medication delivery device or another component of a medication delivery system. Additional information regarding medication delivery devices and/or systems, such as, for example, integrated systems, is provided in U.S. Patent Application Publication No. US2006/0224141, published on Oct. 5, 2006, entitled “Method and System for Providing Integrated Medication Infusion and Analyte Monitoring System”, and U.S. Patent Application Publication No. US2004/0254434, published on Dec. 16, 2004, entitled “Glucose Measuring Module and Insulin Pump Combination,” the disclosure of each of which is incorporated by reference herein in its entirety. Medication delivery devices which may be provided with analyte measurement system as described herein include, e.g., a needle, syringe, pump, catheter, inhaler, transdermal patch, or combination thereof. In some embodiments, the medication delivery device or system may be in the form of a drug delivery injection pen such as a pen-type injection device incorporated within the housing of an analyte measurement system. Additional information is provided in U.S. Pat. Nos. 5,536,249 and 5,925,021, the disclosures of each of which are incorporated by reference herein in their entirety.

### Communication Interface

As discussed previously herein, an analyte measurement system according to the present disclosure can be configured to include a communication interface. In some embodiments, the communication interface includes a receiver and/or transmitter for communicating with a network and/or another device, e.g., a medication delivery device and/or a patient monitoring device, e.g., a continuous glucose monitoring device. In some embodiments, the communication interface is configured for communication with a health management system, such as the CoPilot™ system available from Abbott Diabetes Care Inc., Alameda, Calif.

The communication interface can be configured for wired or wireless communication, including, but not limited to, radio frequency (RF) communication (e.g., Radio-Frequency Identification (RFID), Zigbee communication protocols, WiFi, infrared, wireless Universal Serial Bus (USB), Ultra Wide Band (UWB), Bluetooth® communication protocols, and cellular communication, such as code division multiple access (CDMA) or Global System for Mobile communications (GSM).

In one embodiment, the communication interface is configured to include one or more communication ports, e.g., physical ports or interfaces such as a USB port, an RS-232 port, or any other suitable electrical connection port to allow data communication between the analyte measurement system and other external devices such as a computer terminal (for example, at a physician's office or in hospital environment), an external medical device, such as an infusion device or including an insulin delivery device, or other devices that are configured for similar complementary data communication.

In one embodiment, the communication interface is configured for infrared communication, Bluetooth® communication, or any other suitable wireless communication protocol to enable the analyte measurement system to communicate with other devices such as infusion devices, analyte monitoring devices, computer terminals and/or networks, communication enabled mobile telephones, personal digital assistants, or any other communication devices which the patient or user of the analyte measurement system may use in conjunction therewith, in managing the treatment of a health condition, such as diabetes.

In one embodiment, the communication interface is configured to provide a connection for data transfer utilizing Internet Protocol (IP) through a cell phone network, Short Message Service (SMS), wireless connection to a personal computer (PC) on a Local Area Network (LAN) which is connected to the internet, or WiFi connection to the internet at a WiFi hotspot.

In one embodiment, the analyte measurement system is configured to wirelessly communicate with a server device via the communication interface, e.g., using a common standard such as 802.11 or Bluetooth® RF protocol, or an IrDA infrared protocol. The server device could be another portable device, such as a smart phone, Personal Digital Assistant (PDA) or notebook computer; or a larger device such as a desktop computer, appliance, etc. In some embodiments, the server device has a display, such as a liquid crystal display (LCD), as well as an input device, such as buttons, a keyboard, mouse or touch-screen. With such an arrangement, the user can control the analyte measurement system indirectly by interacting with the user interface(s) of the server device, which in turn interacts with the analyte measurement system across a wireless link.

In some embodiments, the communication interface is configured to automatically or semi-automatically communicate data stored in the analyte measurement system, e.g., in an optional data storage unit, with a network or server device using one or more of the communication protocols and/or mechanisms described above.

### Analytes

A variety of analytes can be detected and quantified using the disclosed analyte measurement system. Analytes that may be determined include, for example, acetyl choline, amylase, bilirubin, cholesterol, chorionic gonadotropin, creatine kinase (e.g., CK-MB), creatine, DNA, fructosamine, glucose, glutamine, growth hormones, hormones, ketones (e.g., ketone bodies), lactate, oxygen, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, may also be determined. Assays suitable for determining the concentration of DNA and/or RNA are disclosed in U.S. Pat. No. 6,281,006 and U.S. Pat. No. 6,638,716, the disclosures of each of which are incorporated by reference herein in their entirety.

### Conclusion

The foregoing description of the invention has been presented for purposes of illustration and description. It is not intended to be exhaustive or to limit the invention to the precise form disclosed. Other modifications and variations may be possible in light of the above teachings. The embodiments were chosen and described in order to best explain the principles of the invention and its practical application, and to thereby enable others skilled in the art to best utilize the invention in various embodiments and various modifications as are suited to the particular use contemplated. It is intended that the appended claims be construed to include other alternative embodiments of the invention; including equivalent structures, components, methods, and means.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In the description of the invention herein, it will be understood that a word appearing in the singular encompasses its plural counterpart, and a word appearing in the plural encompasses its singular counterpart, unless implicitly or explicitly understood or stated otherwise. Merely by way of example, reference to “an” or “the” “analyte” encompasses a single analyte, as well as a combination and/or mixture of two or more different analytes, reference to “a” or “the” “concentration value” encompasses a single concentration value, as well as two or more concentration values, and the like, unless implicitly or explicitly understood or stated otherwise. Further, it will be understood that for any given component described herein, any of the possible candidates or alternatives listed for that component, may generally be used individually or in combination with one another, unless implicitly or explicitly understood or stated otherwise. Additionally, it will be understood that any list of such candidates or alternatives, is merely illustrative, not limiting, unless implicitly or explicitly understood or stated otherwise.

Various terms are described herein to facilitate an understanding of the invention. It will be understood that a corresponding description of these various terms applies to corresponding linguistic or grammatical variations or forms of these various terms. It will also be understood that the invention is not limited to the terminology used herein, or the descriptions thereof, for the description of particular embodiments. Merely by way of example, the invention is not limited to particular analytes, bodily or tissue fluids, blood or capillary blood, or sensor constructs or usages, unless implicitly or explicitly understood or stated otherwise, as such may vary.

The publications discussed herein are provided solely for their disclosure prior to the filing date of the application. Nothing herein is to be construed as an admission that the embodiments of the invention are not entitled to antedate such publication by virtue of prior invention. Further, the dates of publication provided may be different from the actual publication dates which may need to be independently confirmed.

It is to be appreciated that the Detailed Description section, and not the Summary and Abstract sections, is intended to be used to interpret the claims. The Summary and Abstract sections may set forth one or more, but not all exemplary embodiments of the present invention as contemplated by the inventor(s), and thus, are not intended to limit the present invention and the appended claims in any way.

What is claimed is:

1. An analyte monitoring system, comprising:
an on-body housing;
an analyte sensor coupled to the housing, wherein a portion of the analyte sensor extends from the housing for implantation into a patient's body;
an electrical output interface disposed on an outer surface of the housing, wherein the electrical output interface is electrically coupled to the analyte sensor; and
a removable adaptor that mechanically engages with the housing and electrically couples to the electrical output interface, wherein the removable adaptor serves as a data conduit between the analyte sensor and a remote device.

2. The analyte monitoring system of claim 1, wherein the removable adaptor includes a memory unit for logging analyte concentration data received from the implantable analyte sensor.

3. The analyte monitoring system of claim 1, wherein the removable adaptor includes a communications unit for transmitting data to an external receiver.

4. The analyte monitoring system of claim 3, wherein the communications unit transmits the data wireles sly.

5. The analyte monitoring system of claim 4, wherein the wireless transmission is conducted via radio frequency, Bluetooth, ZigBee, infra-red, or other near-field wireless communication protocol.

6. The analyte monitoring system of claim 1, wherein the removable adaptor is a circular shape.

7. The analyte monitoring system of claim 1, wherein the removable adaptor is shaped such that its connection to the housing and electrical output interface has no orientational preference.

8. The analyte monitoring system of claim 1, wherein the removable adaptor includes an elongated data cord extending from the housing.

9. The analyte monitoring system of claim 8, wherein the elongated data cord includes a data cord output interface for direct coupling to the remote device.

10. The analyte monitoring system of claim 8, wherein the elongated data cord includes a communications unit for wirelessly transmitting data from the analyte sensor to the remote device.

11. The analyte monitoring system of claim 1, wherein the data is glucose concentration data.

12. The analyte monitoring system of claim 1, wherein the data is ketone concentration data.

13. The analyte monitoring system of claim 1, wherein the removable adaptor serves as a data conduit that transmits an instantaneous data reading upon request from the remote device.

14. An analyte monitoring system, comprising:
an on-body housing;
an analyte sensor coupled to the housing, wherein a portion of the analyte sensor extends from the housing for implantation into a patient's body;
an electrical output interface disposed on an outer surface of the housing, wherein the electrical output interface is electrically coupled to the analyte sensor; and
a removable adaptor that mechanically engages with the housing and electrically couples to the electrical output interface, wherein the removable adaptor serves as a data conduit between the analyte sensor and a remote device, wherein the removable adaptor is shaped such that its connection to the housing and electrical output interface has no orientational preference, and wherein the removable adaptor includes
a memory unit for logging analyte concentration data received from the implantable analyte sensor,
a communications unit for transmitting data to the remote device.

15. The analyte monitoring system of claim 14, wherein the communications unit transmits the data wireles sly.

16. The analyte monitoring system of claim 15, wherein the wireless transmission is conducted via radio frequency, Bluetooth, ZigBee, infra-red, or other near-field wireless communication protocol.

17. The analyte monitoring system of claim 14, wherein the data is glucose concentration data.

18. The analyte monitoring system of claim 14, wherein the data is ketone concentration data.

19. An analyte monitoring system, comprising:
an on-body housing;
an analyte sensor coupled to the housing, wherein a portion of the analyte sensor extends from the housing for implantation into a patient's body;
an electrical output interface disposed on an outer surface of the housing, wherein the electrical output interface is electrically coupled to the analyte sensor; and
a removable data cord that mechanically engages with the housing and electrically couples to the electrical output interface, wherein the data cord extends from the housing and serves as a data conduit between the analyte sensor and a remote device.

20. The analyte monitoring system of claim 19, wherein the data cord includes a communications unit for transmitting data to an external receiver.

21. The analyte monitoring system of claim 20, wherein the communications unit transmits the data wireles sly.

22. The analyte monitoring system of claim 21, wherein the wireless transmission is conducted via radio frequency, Bluetooth, ZigBee, infra-red, or other near-field wireless communication protocol.

23. The analyte monitoring system of claim 19, wherein the data cord includes a data cord output interface for direct coupling to the remote device.

24. The analyte monitoring system of claim 19, wherein the data is glucose concentration data.

25. The analyte monitoring system of claim 19, wherein the data is ketone concentration data.

26. The analyte monitoring system of claim 19, wherein the data cord serves as a data conduit that transmits an instantaneous data reading upon request from the remote device.

27. An analyte monitoring system, comprising:
an on-body housing;
a self-powered analyte sensor coupled to the housing, wherein a portion of the analyte sensor extends from the housing for implantation into a patient's body;
an electrical output interface disposed on an outer surface of the housing, wherein the electrical output interface is electrically coupled to the analyte sensor; and
a removable adaptor that mechanically engages with the housing and electrically couples to the electrical output interface, wherein the removable adaptor serves as a data conduit between the analyte sensor and a remote device.

28. The analyte monitoring system of claim 27, wherein the removable adaptor includes a memory unit for logging analyte concentration data received from the implantable analyte sensor.

29. The analyte monitoring system of claim 27, wherein the removable adaptor includes a communications unit for transmitting data to an external receiver.

30. The analyte monitoring system of claim 27, wherein the communications unit transmits the data wireles sly.

31. The analyte monitoring system of claim 30, wherein the wireless transmission is conducted via radio frequency, Bluetooth, ZigBee, infra-red, or other near-field wireless communication protocol.

32. The analyte monitoring system of claim 27, wherein the removable adaptor is a circular shape.

33. The analyte monitoring system of claim 27, wherein the removable adaptor is shaped such that its connection to the housing and electrical output interface has no orientational preference.

34. The analyte monitoring system of claim 27, wherein the removable adaptor includes an elongated data cord extending from the housing.

35. The analyte monitoring system of claim 34, wherein the elongated data cord includes a data cord output interface for direct coupling to the remote device.

36. The analyte monitoring system of claim 34, wherein the elongated data cord includes a communications unit for wirelessly transmitting data from the analyte sensor to the remote device.

37. The analyte monitoring system of claim 27, wherein the data is glucose concentration data.

38. The analyte monitoring system of claim 27, wherein the data is ketone concentration data.

39. The analyte monitoring system of claim 27, wherein the removable adaptor serves as a data conduit that transmits an instantaneous data reading upon request from the remote device.

40. A method of preparing an analyte monitoring system, comprising:
sterilizing a self-powered analyte sensor by electron beam sterilization;
coupling the analyte sensor to an on-body housing, wherein a portion of the analyte sensor extends from the housing for implantation into a patient's body;
electrically coupling an electrical output interface disposed on an outer surface of the housing to the analyte sensor;
sterilizing a removable adaptor unit with ethylene oxide; and
mechanically coupling the adaptor to the housing and electrically coupling the adaptor to the electrical output interface, wherein the removable adaptor serves as a data conduit between the analyte sensor and a remote device.
